(19) United States
(12) Patent Application Publication
(10) Pub. No.: US 2010/0034765 A1
(43) Pub. Date: Feb. 11, 2010

(54) USE OF POLYSILOXANES WITH QUATERNARY AMMONIUM GROUPS FOR PROTECTING ANIMAL OR HUMAN HAIR AGAINST HEAT DAMAGE

(75) Inventors: Sascha Herrwerth, Essen (DE); Michael Ferenz, Essen (DE); Patrick Winter, Muelheim an der Ruhr (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/536,146

(22) Filed: Aug. 5, 2009

(30) Foreign Application Priority Data
Aug. 6, 2008 (DE) 102008041020.9

(51) Int. Cl. A61K 8/898; A61Q 5/00; C08G 77/388

(52) U.S. Cl. 424/70.122; 525/474

(57) ABSTRACT

The invention relates to the use of polysiloxanes containing at least one quaternary ammonium group for protecting animal or human hair against heat damage.

### FIELD OF THE INVENTION

The invention relates to the use of polysiloxanes containing at least one quaternary ammonium group for protecting animal or human hair against heat damage.

### BACKGROUND OF THE INVENTION

Human hair is exposed daily to a very wide variety of influences. Besides mechanical stresses as a result of brushing, combing, putting the hair up or tying the hair back, the hair is also attacked by environmental influences such as, for example, strong UV radiation, cold, heat, wind and water. The physiological status (e.g., age, health) of the particular person also influences the condition of the keratin fibers.

In particular, treatment with chemical agents changes the structure and surface properties of the hair. Methods such as, for example, permanent waving, bleaching, coloring, tinting, straightening etc., but also frequent washing with aggressive surfactants contribute to greater or lesser damage being caused to the hair structure. Thus, for example, during a permanent wave, both the cortex and also the cuticle of the hair are attacked. The disulfide bridges of the cystein are broken by the reduction step and, in the subsequent oxidation step, are in part oxidized to cysteic acid.

The treatment of head hair for cleaning, coloring, waving, straightening, setting or shaping is carried out predominantly with aqueous preparations. After these treatment methods, the hair is dried again. In order to achieve this in a short time, drying is carried out using hot air. The use of hot-air hairdryers however, also serves to intensify the shaping and the setting treatment. Curling tongs are also used with the aim of permanently setting hair curling.

Furthermore, smoothing irons are used for straightening and setting hair; these can generate temperatures greater than 200° C.

The specified methods of hot-air and heat applications for drying and shaping damage the structure of hair.

Attention has been paid to the problem of hair damage caused by the effect of heat for a long time, and numerous proposals have already been made to reduce the problem of thermal heat damage through additives to hair treatment compositions.

WO 00/44337 discloses the use of copolymers of polysiloxane and proteins in cosmetic formulations to protect hair from damage by heat treatments. Similarly, structures are also described in the article “Test method for the study of ingredients protecting hair against damage caused by thermal stresses” by S. Meulemann (SÖFW-Journal 128th volume, 3, 2008).

U.S. Pat. No. 6,241,977 describes copolymers of vinyl ether and maleic acid as active ingredients which protect hair and wool against damage by heat in the range from 100-180° C.

DE 19943597 discloses the use of basic amino acids in aqueous hair treatment compositions as blow-drying protection of the hair. L-histidine is preferably suitable in those formulations which serve to shape and set the hair with the use of hot air.

JP 03157316, for example, proposes using a combination of quaternary ammonium salts, certain plant extracts and water-soluble polymers with quaternary ammonium groups.

JP 03135909 proposes the use of high molecular weight silicones and fatty acid alkanolamides.

According to WO 99/11224, a combination of a fiber-structure-improving active ingredient such as, for example, panthenol, and a conditioning active ingredient such as, for example, a cationic surfactant, is suitable for preventing heat damage of the hair.

There is thus still a need for versatile active ingredients for cleaning and care formulations for hair such as, for example, shampoos, leave-in conditioners, hair rinses and hair after-treatment compositions, which, besides the cleaning and care effect, effectively protect the hair against damage due to the effect of heat, and minimize the change in hair structure.

Polysiloxanes with quaternary groups and their use as additives for hair care or textile softeners are known from the patent literature.

Thus, for example, DE 14 93 384, EP 0017122 and U.S. Pat. No. 4,895,964 describe structures in which siloxanes are modified in the middle with ammonium groups distributed randomly over the polymer. These compounds have the disadvantage that a marked silicone character is diminished and good effectiveness can no longer be observed.

Cationic polysiloxanes as described in DE 37 19 086 and EP 0 294 642 have a more marked silicone character. Those in DE 37 19 086 and those in EP 0 294 642 describe structures in which the quaternary functions are terminally bonded to the polysiloxane. Compounds of this type offer advantages in regard to their effect as conditioners both for hair and textiles and also for hard surfaces. The use of such compounds in cosmetic formulations has been described, for example, in EP 0 530 974, EP 617 607, EP 1080714, WO 2001082879 and U.S. Pat. No. 6,207,141.

EP 1887024 describes terminally cationic polysiloxanes with a T-structure and their use as conditioners in cosmetic formulations. These cationic polysiloxanes exhibit a marked conditioning and shine-generating effect.

None of the cited references describes an influence of the polysiloxanes containing quaternary ammonium groups on the damage which the hair suffers due to the effect of heat.

### SUMMARY OF THE INVENTION

The present invention provides an active ingredient which is able to protect the hair significantly against damage due to the effect of heat.

In the search for active ingredients which protect the hair against heat damage, it has surprisingly been found that cationic polysiloxanes develop a significant protective effect against the effect of heat for the hair keratin.

The invention therefore provides the use of polysiloxanes containing at least one quaternary ammonium group for protecting animal or human hair against heat damage. That is, the present invention provides a method for protecting animal or human hair against heat damage in which a polysiloxane containing at least one quaternary ammonium group is employed.

One advantage of the use according to the invention is that these polysiloxanes contribute to improved foaming behavior, increased foam volume and better foam creaminess of the formulations.

A further advantage of the use according to the invention is that the polysiloxanes with quaternary functions can exert exceptional conditioning effects on skin and hair. As a result of this conditioning effect on, for example, the skin, a dry, harsh or rough condition of the skin can be prevented following applications of an aqueous, surface-active formulation, and a pleasant, velvety-silky skin feel is achieved.

Yet a further advantage of the use according to the invention is that properties such as combability, softness, volume, shapeability, shine, manageability and the detangleability of undamaged and damaged hair is improved.

### BRIEF DESCRIPTION OF THE DRAWINGS

FIG. 1 is a graph summarizing DSC measurement results obtained using formulations in accordance with the present invention and DSC measurement results for reference formulations that are outside the scope of the present invention.

### DETAILED DESCRIPTION OF THE INVENTION

The present invention, which provides a method for protecting animal or human hair against heat damage using a formulation including polysiloxanes with quaternary ammonium groups, will now be described in greater detail.

Within the context of the present invention, the term “quaternary ammonium group” contains a monovalently positively charged group with a tetravalent nitrogen.

An example which may be mentioned and employed in the present invention is the following general formula

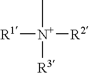

where R^{1′}, R^{2′} and R^{3′}, independently of one another, are identical or different, branched or unbranched, optionally substituted hydrocarbon radicals, which optionally contain ester, amide or ether functions, or H.

Within the context of the present invention, the term “terminally bonded to the polysiloxane” denotes that the quaternary ammonium group is bonded to the siloxane backbone via a so-called M′ unit of the general formula XSiY₂O_{1/2}. X and Y are defined as stated below (see formula 1).

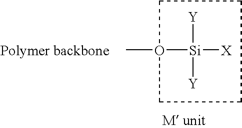

Within the context of the present invention, the term “water-insoluble” is defined as a solubility of less than 0.01% by weight in aqueous solution at 20° C. and a pressure of 1 bar.

Within the context of the present invention, the term “water-soluble” is defined as a solubility of equal to or more than 0.01% by weight in an aqueous solution at 20° C. and a pressure of 1 bar.

If, within the context of the present invention, compounds such as, for example, polysiloxanes, are described which can have different units several times, then these units may occur in random distribution (random oligomer) or they may be arranged (block oligomer) in these compounds. Details relating to the number of units in such compounds are to be understood as meaning an average value, averaged over all of the corresponding compounds.

Unless stated otherwise, all of the percent (%) stated are percent by mass.

Unless stated otherwise, all of the conditions such as, for example, pressure and temperature are standard conditions.

The heat damage in which protection is sought can be caused by all heat sources. The heat affecting the hair can act on the hair, for example, directly or through radiation or through convection. Examples of everyday devices which cause heat to act upon hair include: e.g., hot-air hairdryers, smoothing irons and curling tongs.

The heat may also be of natural origin, e.g., solar irradiation.

According to the invention, all polysiloxanes containing at least one quaternary ammonium group can be used.

Preferably, the polysiloxanes used in the invention are those whose overall charge is positive and are thus cationic polysiloxanes.

Water-soluble or water-insoluble polysiloxanes can be used. Depending on the formulation to be produced (opaque or clear formulations), it is known to a person skilled in the art whether water-soluble or insoluble polysiloxanes should be used to produce the formulation.

Preferably, the polysiloxane used in the invention contains at least two, preferably at least three, quaternary ammonium groups.

According to the invention, preference is given to using polysiloxanes in which at least one quaternary ammonium group is terminally bonded to the polysiloxane. In one embodiment of the invention it is preferred to use polysiloxanes that contain in total exactly two quaternary ammonium groups bonded terminally to the polysiloxane. Examples of such polysiloxanes can be found in DE 37 19 086 and EP 0 294 642.

Moreover, it is highly preferred in the present invention to use polysiloxanes of the general formula I.

[M′Dₙ]₃T Formula I

wherein:
M′=XSiY₂O_{1/2}
D=SiY₂O_{2/2}
T=SiZO_{3/2}
X are identical or different organic radicals which carry quaternary ammonium groups,
Y are identical or different radicals from the group alkyl, aryl or alkaryl having 1 to 30 carbon atoms, preferably methyl or phenyl, in particular methyl,
Z are identical or different radicals from the group alkyl, aryl or alkaryl having 1 to 30 carbon atoms, preferably methyl or phenyl,
n is 2 to 200, preferably 3 to 120, in particular 8 to 80.

Suitable radicals X are, for example, groups with the structure —R1-R2, in which
R1 are preferably identical or different divalent radicals selected from the group

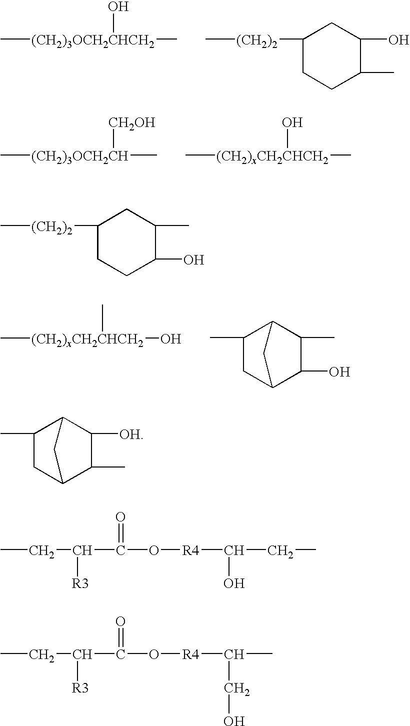

R1 is preferably:

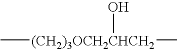

R2 is selected from the group consisting of

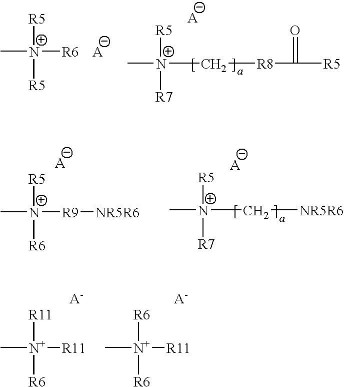

R3 are identical or different radicals from the group hydrogen or alkyl having 1 to 6 carbon atoms, preferably methyl,
R4 are identical or different divalent hydrocarbon radicals which optionally contain ether functions, preferably methylene,
R5, R6, R7 are in each case independently of one another hydrogen or alkyl radicals having 1 to 30 carbon atoms,
R8 are identical or different radicals from the group —O—; —NR10,
R9 are identical or different optionally branched divalent hydrocarbon radicals,
R10 are identical or different radicals from the group hydrogen or alkyl having 1 to 6 carbon atoms,
R11 are identical or different radicals of the general formula:

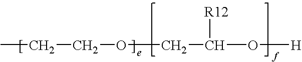

R12 are identical or different alkyl, aryl or alkaryl radicals having 1 to 30 carbon atoms which optionally contain ether functions, preferably methyl, ethyl or phenyl, in particular methyl,
e is 0 to 20, preferably 0 to 10, in particular 1 to 3,
f is 0 to 20, preferably 0 to 10,

e+f is >=1,
x is 2 to 18,
a is 2 to 18, preferably 3,
A⁻ are identical or different counterions to the positive charges on the quaternary ammonium groups selected from inorganic or organic anions of the acids HA.

Examples of such polysiloxanes are described in EP 1887024.

One advantage of using the polysiloxanes according to formula I is a reduced viscosity-lowering effect compared to the cationic polysiloxanes, as described in DE 37 19 086 and EP 0 294 642. This leads to a significant reduction in thickener required in order to adjust the formulations to the desired viscosity. This, in turn, allows a simplification of the formulation which takes into account the aspect of preserving resources.

The use according to the invention of the polysiloxanes normally takes place in the form of application of cosmetic formulations.

Preferably, the cosmetic formulations are cleaning and care formulations.

Cleaning and care formulations are understood as meaning primarily those formulations for the treatment of hair following whose application the hair is usually treated with hot-air hairdryers or smoothing irons. Such hair treatment compositions are, for example, hair shampoos, liquid soaps, hair rinses, permanent wave neutralizing lotions, hair color shampoos, hair setting compositions, hair arranging compositions, hair styling preparations, blow-drying lotions, foam setting compositions, hair treatments, leave-in conditioners and other cleaning and care formulations.

The present invention thus also further provides the use of polysiloxanes comprising at least one quaternary ammonium group for producing cosmetic formulations for protecting human or animal hair against heat damage.

In the preparation of the cosmetic formulations for protecting human or animal hair against heat damage, the groups of polysiloxanes described above, preferably the groups of polysiloxanes described above as preferred, are used.

The polysiloxanes containing at least one quaternary ammonium group are used in a concentration of from 0.01 to 20% by mass, preferably 0.1 to 8% by mass, particularly preferably from 0.2 to 4% by mass, in the cosmetic formulations.

The present invention thus also further provides cosmetic formulations for protecting human or animal hair against heat damage which are obtained by using the polysiloxanes containing at least one quaternary ammonium group for producing the formulations.

The cosmetic formulations according to the invention for protecting human or animal hair against heat damage are preferably obtained by using the groups of polysiloxanes described above, in particular the groups of polysiloxanes described above as preferred, for producing the formulations.

The cosmetic formulations for protecting human or animal hair against heat damage comprising polysiloxanes containing quaternary ammonium groups can comprise, for example, at least one additional component, selected from the group of
emollients,
emulsifiers and surfactants,
thickeners/viscosity regulators/stabilizers,
UV photoprotective filters,
antioxidants and vitamins,
hydrotropes (or polyols),
solids and fillers,
film formers,
pearlescent additives,
deodorant and antiperspirant active ingredients,
insect repellents,
self-tanning agents,
preservatives,
conditioners,
perfumes,
dyes,
biogenic active ingredients,
care additives,
superfatting agents,
solvents.

Emollients which can be used are all cosmetic oils, in particular mono- or diesters of linear and/or branched mono- and/or dicarboxylic acids having 2 to 44 carbon atoms with linear and/or branched saturated or unsaturated alcohols having 1 to 22 carbon atoms. It is also possible to use the esterification products of aliphatic, difunctional alcohols having 2 to 36 carbon atoms with monofunctional aliphatic carboxylic acids having 1 to 22 carbon atoms. Also suitable are long-chain aryl acid esters, such as, for example, esters of benzoic acid, e.g., benzoic acid esters of linear or branched, saturated or unsaturated alcohols having 1 to 22 carbon atoms, or else isostearyl benzoate or octyldodecyl benzoate. Further monoesters suitable as emollients and oil components are, for example, the methyl esters and isopropyl esters of fatty acids having 12 to 22 carbon atoms, such as, for example, methyl laurate, methyl stearate, methyl oleate, methyl erucate, isopropyl palmitate, isopropyl myristate, isopropyl stearate, isopropyl oleate. Other suitable monoesters are, for example, n-butyl stearate, n-hexyl laurate, n-decyl oleate, isooctyl stearate, isononyl palmitate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, and esters which are obtainable from technical-grade aliphatic alcohol cuts and technical-grade, aliphatic carboxylic acid mixtures, e.g., esters of unsaturated fatty alcohols, having 12 to 22 carbon atoms and saturated and unsaturated fatty acids having 12 to 22 carbon atoms, as are accessible from animal and vegetable fats. Also suitable, however, are naturally occurring monoester and/or wax ester mixtures, as are present, for example in jojoba oil or in sperm oil. Suitable dicarboxylic acid esters are, for example, di-n-butyl adipate, di-n-butyl sebacate, di(2-ethylhexyl)adipate, di(2-hexyldecyl)succinate, diisotridecyl azelate. Suitable diol esters are, for example, ethylene glycol dioleate, ethylene glycol diisotridecanoate, propylene glycol di(2-ethylhexanoate), butanediol diisostearate, butanediol dicaprylate/caprate and neopentyl glycol dicaprylate. Further fatty acid esters which can be used as emollients are, for example, C₁₂₋₁₅ alkyl benzoate, dicaprylyl carbonate, diethylhexyl carbonate. Emollients and oil components which can likewise be used are longer-chain triglycerides, i.e., triple esters of glycerol with three acid molecules, of which at least one is relatively long-chain. By way of example, mention may be made here of fatty acid triglycerides; examples of such which may be used are natural, vegetable oils, e.g., olive oil, sunflower oil, soybean oil, peanut oil, rapeseed oil, almond oil, sesame oil, avocado oil, castor oil, cocoa butter, palm oil, but also the liquid fractions of coconut oil or of palm kernel oil, and also animal oils, such as, for example, shark liver oil, cod liver oil, whale oil, beef tallow and butter fat, waxes such as beeswax, carnauba palm wax, spermaceti, lanolin and claw oil, the liquid fractions of beef tallow and also synthetic triglycerides of capryl/capric acid mixtures, triglycerides of technical-grade oleic acid, triglycerides with isostearic acid, or from palmitic acid/oleic acid mixtures as emollients and oil components. Furthermore, hydrocarbons, in particular also liquid paraffins and isoparaffins, can be used. Examples of hydrocarbons which can be used are paraffin oil, isohexadecane, polydecene, vaseline, Paraffinum perliquidum, squalane, ceresine. Furthermore, it is also possible to use linear or branched fatty alcohols such as oleyl alcohol or octyldodecanol, and also fatty alcohol ethers such as dicaprylyl ether. Suitable silicone oils and silicone waxes are, for example, polydimethylsiloxanes, cyclomethylsiloxanes, and also aryl- or alkyl- or alkoxy-substituted polymethylsiloxanes or cyclomethylsiloxanes. Suitable further oil bodies are, for example, Guerbet alcohols based on fatty alcohols having 6 to 18, preferably 8 to 10, carbon atoms, esters of linear C6-C22-fatty acids with linear C6- C22-fatty alcohols, esters of branched C6-C13-carboxylic acids with linear C6-C22-fatty alcohols, esters of linear C6-C22-fatty acids with branched C8-C 18-alcohols, in particular 2-ethylhexanol or isononanol, esters of branched C6-C13-carboxylic acids with branched alcohols, in particular 2-ethylhexanol or isononanol, esters of linear and/or branched fatty acids with polyhydric alcohols (such as, for example, propylene glycol, dimerdiol or trimertriol) and/or Guerbet alcohols, triglycerides based on C6-C10-fatty acids, liquid mono-/di-/triglyceride mixtures based on C6-C18-fatty acids, esters of C6-C22-fatty alcohols and/or Guerbet alcohols with aromatic carboxylic acids, in particular benzoic acid, vegetable oils, branched primary alcohols, substituted cyclohexanes, linear C6-C22-fatty alcohol carbonates, Guerbet carbonates, esters of benzoic acid with linear and/or branched C6-C22-alcohols (e.g. Finsolv™ TN), dialkyl ethers, ring-opening products of epoxidized fatty acid esters with polyols, silicone oils and/or aliphatic or naphthenic hydrocarbons.

Emulsifiers or surfactants which may be used are non-ionic, anionic, cationic or amphoteric surfactants.

Nonionogenic emulsifiers or surfactants which can be used are compounds from at least one of the following groups:
addition products of from 2 to 100 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C_{12/18}-fatty acid mono- and diesters of addition products of from 1 to 100 mol of ethylene oxide onto glycerol,
glycerol mono- and diesters and sorbitan mono- and diesters of saturated and unsaturated fatty acids having 6 to 22 carbon atoms and ethylene oxide addition products thereof,
alkyl mono- and oligoglycosides having 8 to 22 carbon atoms in the alkyl radical and ethylene oxide addition products thereof,
addition products of from 2 to 200 mol of ethylene oxide onto castor oil and/or hydrogenated castor oil,
partial esters based on linear, branched, unsaturated or saturated C₆-C₂₂-fatty acids, ricinoleic acid, and 12-hydroxystearic acid and glycerol, polyglycerol, pentaerythritol, dipentaerythritol, sugar alcohols (e.g., sorbitol), alkyl glucosides (e.g., methyl glucoside, butyl glucoside, lauryl glucoside) and polyglucosides (e.g., cellulose),
mono-, di- and trialkylphosphates, and mono-, di- and/or tri-PEG alkyl phosphates and salts thereof,
polysiloxane-polyether copolymers (dimethicone copolyols), such as, for example PEG/PPG-20/6 dimethicone, PEG/PPG-20/20 dimethicone, bis-PEG/PPG-20/20 dimethicone, PEG-12 or PEG-14 dimethicone, PEG/PPG-14/4 or 4/12 or 20/20 or 18/18 or 17/18 or 15/15,
polysiloxane-polyalkyl-polyether copolymers and corresponding derivatives, such as, for example, lauryl or cetyl dimethicone copolyols, in particular cetyl PEG/PPG-10/1 dimethicone (ABIL® EM 90 (Evonik Goldschmidt GmbH)),
mixed esters of pentaerythritol, fatty acids, citric acid and fatty alcohol as in DE 11 65 574 and/or mixed esters of fatty acids having 6 to 22 carbon atoms, methylglucose and polyols, such as, for example, glycerol or polyglycerol, citric acid esters, such as, for example, glyceryl stearate citrate, glyceryl oleate citrate and dilauryl citrate.

Anionic emulsifiers or surfactants can contain water-solubilizing anionic groups, such as, for example, a carboxylate, sulphate, sulphonate or phosphate group and a lipophilic radical. Skin-compatible anionic surfactants are known to a person skilled in the art in large numbers and are commercially available. Here, these may be alkyl sulphates or alkyl phosphates in the form of their alkali metal, ammonium or alkanolammonium salts, alkyl ether sulphates, alkyl ether carboxylates, acyl sarcosinates, and sulphosuccinates and acyl glutamates in the form of their alkali metal or ammonium salts.

Cationic emulsifiers and surfactants can also be added. Those which can be used are, in particular, quaternary ammonium compounds, in particular those provided with at least one linear and/or branched, saturated or unsaturated alkyl chain having 8 to 22 carbon atoms, such as, for example, alkyltrimethylammonium halides, such as, for example, cetyltrimethylammonium chloride or bromide or behenyltrimethylammonium chloride, but also dialkyldimethylammonium halides, such as, for example, distearyldimethylammonium chloride.

Furthermore, monoalkylamidoquats such as, for example, pal-mitamidopropyltrimethylammonium chloride or corresponding dialkylamidoquats, can be used.

Furthermore, readily biodegradable quaternary ester compounds can be used; these may be quaternized fatty acid esters based on mono-, di- or triethanolamine. Furthermore, alkylguanidinium salts can be added as cationic emulsifiers.

Typical examples of mild, i.e. particularly skin-compatible, surfactants are fatty alcohol polyglycol ether sulphates, monoglyceride sulphates, mono- and/or dialkyl sulphosuccinates, fatty acid isethionates, fatty acid sarcosinates, fatty acid taurides, fatty acid glutamates, ether carboxylic acids, alkyl oligoglucosides, fatty acid glucamides, alkylamidobetaines and/or protein fatty acid condensates, the latter for example based on wheat proteins.

Furthermore, it is possible to use amphoteric surfactants, such as, for example, betaines, amphoacetates or amphopropionates, thus, for example, substances such as the N-alkyl-N,N-dimethylammonium glycinates, for example cocoalkyldimethylammonium glycinate, N-acylaminopropyl-N,N-dimethylammonium glycinates, for example cocoa-cylaminopropyldimethylammonium glycinate, and 2-alkyl-3-carboxymethyl-3-hydroxyethyl-imidazolines having in each case 8 to 18 carbon atoms in the alkyl or acyl group, and also cocoacylaminoethyl hydroxyethylcarboxymethyl glycinate.

Of the ampholytic surfactants, it is possible to use those surface-active compounds which, apart from a C8/18-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkyl-amidopropylglycines, N-alkyltaurines, N-alkylsarcosines, 2-alkylaminopropionic acids and alkylaminoacetic acids having in each case about 8 to 18 carbon atoms in the alkyl group. Further examples of ampholytic surfactants are N-cocoalkylaminopropionate, cocoacylaminoethylaminopropionate and C12/18-acylsarcosine.

Suitable thickeners are, for example, polysaccharides, in particular xanthan gum, guar-guar, agar agar, alginates and tyloses, carboxymethylcellulose and hydroxyethylcellulose, also relatively high molecular weight polyethylene glycol mono- and diesters of fatty acids, polyacrylates (e.g., Carbopols™ or Synthalens™), polyacrylamides, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates with a narrowed homologue distribution or alkyl oligoglucosides, and also electrolytes such as sodium chloride and ammonium chloride.

Suitable thickeners for thickening oil phases are all thickeners known to a person skilled in the art. In particular, mention is to be made here of waxes, such as hydrogenated castor wax, beeswax or microwax. Furthermore, inorganic thickeners can also be used, such as silica, alumina or sheet silicates (e.g., hectorite, laponite, saponite). In this connection, these inorganic oil phase thickeners may be hydrophobically modified. For the thickening/stabilization of water-in-oil emulsions, in particular aerosils, sheet silicates and/or metal salts of fatty acids, such as, for example, zinc stearate, can be used here.

Viscosity regulators for aqueous surfactant systems which may be present are, for example, NaCl, low molecular weight non-ionic surfactants, such as cocoamide DEA/MEA and laureth-3, or polymeric, high molecular weight, associative, highly ethoxylate fat derivatives, such as PEG-200 hydrogenated glyceryl palmate.

UV photoprotective filters which can be used are, for example, organic substances which are able to absorb ultraviolet rays and which give off the absorbed energy again in the form of longer-wave radiation, e.g., heat. UVB filters may be oil-soluble or water-soluble. Examples of oil-soluble UVB photoprotective filters are:
3-benzylidenecamphor and derivatives thereof, e.g., 3-(4-methylbenzylidene)camphor,
4-aminobenzoic acid derivatives, such as, for example, 2-ethylhexyl 4-(dimethylamino)benzoate, 2-ethylhexyl 4-(dimethylamino)benzoate and amyl 4-(dimethylamino)benzoate,
esters of cinnamic acid, such as 2-ethylhexyl 4-methoxycinnamate, isopentyl 4-methoxycinnamate, 2-ethylhexyl 2-cyano-3-phenylcinnamate(octocrylene),
esters of salicylic acid, such as, for example, 2-ethylhexyl salicylate, 4-isopropylbenzyl salicylate, homomenthyl salicylate,
derivatives of benzophenone, such as, for example, 2-hydroxy-4-methoxybenzophenone, 2-hydroxy-4-methoxy-4′-methylbenzophenone, 2,2′-dihydroxy-4-methoxybenzophenone, esters of benzalmalonic acid, such as, for example, di-2-ethylhexyl 4-methoxybenzmalonate,
triazine derivatives, such as, for example, 2,4,6-trianilino-(p-carbo-2′-ethyl-1′-hexyloxy)-1,3,5-triazine and octyltriazone,
propane-1,3-diones, such as, for example, 1-(4-tert-butylphenyl)-3-(4′-methoxyphenyl)propane-1,3-dione.

Suitable water-soluble UVB photoprotective filters are:
2-phenylbenzimidazole-5-sulphonic acid and the alkali metal, alkaline earth metal, ammonium, alkylammonium, alkanolammonium and glucammonium salts thereof,
sulphonic acid derivatives of benzophenone, such as, for example, 2-hydroxy-4-methoxybenzophenone-5-sulphonic acid and its salts,
sulphonic acid derivatives of 3-benzylidenecamphor, such as, for example, 4-(2-oxo-3-bornylidenemethyl)benzenesulphonic acid and 2-methyl-5-(2-oxo-3-bornylidene)sulphonic acid and salts thereof.

Suitable typical UVA photoprotective filters are in particular derivatives of benzoylmethane, such as, for example, 1-(4′-tert-butylphenyl)-3-(4′-methoxyphenyl)propane-1,3-dione or 1-phenyl-3-(4′-isopropylphenyl)propane-1,3-dione. The UV-A and UV-B filters can of course also be used in mixtures.

Besides the specified soluble substances, insoluble pigments, namely finely disperse metal oxides or salts are also suitable for this purpose, such as, for example, titanium dioxide, zinc oxide, iron oxide, aluminium oxide, cerium oxide, zirconium oxide, silicates (talc), barium sulphate and zinc stearate. The particles here should have an average diameter of less than 100 nm, e.g. between 5 and 50 nm and in particular between 15 and 30 nm. They can have a spherical shape, although it is also possible to use those particles which have an ellipsoidal shape or a shape which deviates in some other way from the spherical form. A relatively new class of photoprotective filters are micronized organic pigments, such as, for example, 2,2′-methylenebis{6-(2H-benzotriazol-2-yl)-4-(1,1,3,3-tetramethylbutyl)phenol} with a particle size of <200 nm, which is obtainable, for example, as 50% strength aqueous dispersion.

Further suitable UV photoprotective filters can be found in the overview by P. Finkel in SÖFW-Journal 122, 543 (1996).

Besides the two aforementioned groups of primary UV photoprotective filters, it is also possible to use secondary photoprotective agents of the antioxidant type which interrupt the photochemical reaction chain which is triggered when UV radiation penetrates into the skin.

Antioxidants and vitamins which can be used are, for example, superoxide-dismutase, tocopherol (vitamin E), tocopherol sorbate, tocopherol acetate, other esters of tocopherol, dibutylhydroxytoluene and ascorbic acid (vitamin C) and its salts, and also derivatives thereof (e.g., magnesium ascorbyl phosphate, sodium ascorbyl phosphate, ascorbyl sorbate), ascorbyl esters of fatty acids, butylated hydroxybenzoic acid and its salts, peroxides, such as, for example, hydrogen peroxide, perborates, thioglycolates, persulphate salts, 6-hydroxy-2,5,7,8-tetramethylchroman-2-carboxylic acid (TROLOX™), gallic acid and its alkyl esters, uric acid and its salts and alkyl esters, sorbic acid and its salts, lipoic acid, ferulic acid, amines (e.g., N,N-diethylhydroxylamine, aminoguanidines), sulphhydryl compounds (e.g., glutathione), dihydroxy-fumaric acid and its salts, glycine pidolate, arginine pilolate, nordihydroguaiaretic acid, bioflavonoids, curcumin, lysine, L-methionine, proline, superoxide dismutase, silymarin, tea extract, grapefruit peel/pip extract, melanin, rosemary extract, thiooctanoic acid, resveratrol, oxyresveratrol, etc.

Hydrotropes which can be used for improving the flow behavior and the application properties are, for example, ethanol, isopropyl alcohol or polyols. Polyols which are suitable here can have 2 to 15 carbon atoms and at least 2 hydroxyl groups. Typical examples are: glycerol alkylene glycols, such as, for example, ethylene glycol, diethylene glycol, propylene glycol, butylene glycol, hexylene glycol, and polyethylene glycols with an average molecular weight of from 100 to 1,000 daltons, technical-grade oligoglycerol mixtures with a degree of self-condensation of from 1.5 to 10, such as, for example, technical-grade diglycerol mixtures with a diglycerol content of from 40 to 50% by weight, methylol compounds, such as in particular trimethylolethane, trimethylolpropane, trimethylolbutane, pentaerythritol and dipentaerythritol, lower alkyl glucosides, in particular those with 1 to 4 carbon atoms in the alkyl radical, such as, for example, methyl and butyl glucoside, sugar alcohols having 5 to 12 carbon atoms, such as, for example, sorbitol or mannitol, sugars having 5 to 12 carbon atoms, such as, for example, glucose or sucrose, amino sugars, such as, for example, glucamine.

Solids which can be used are, for example, iron oxide pigments, titanium dioxide or zinc oxide particles and those additionally specified under “UV protectants”. Furthermore, it is also possible to use particles which lead to special sensory effects, such as, for example, nylon-12, boron nitride, polymer particles such as, for example, polyacrylate or polymethyl acrylate particles or silicone elastomers. Fillers which can be used include starch and starch derivatives, such as tapioca starch, distarch phosphate, aluminium starch or sodium starch, octenyl succinate, and pigments which have neither primarily a UV filter effect nor a coloring effect, for example Aerosils® (CAS No. 7631-86-9).

Examples of film formers which can be used, for example, for improving the water resistance are: polyurethanes, dimethicones, copolyol, polyacrylates or PVP/VA copolymer (PVP=polyvinylpyrrolidone, VA=vinyl acetate). Fat-soluble film formers which can be used are: e.g., polymers based on polyvinylpyrrolidone (PVP), copolymers of polyvinylpyrrolidone, PVP/hexadecene copolymer or the PVP/eicosene copolymer.

Pearlescence additives which can be used in the invention are, for example, glycol distearates or PEG-3 distearate.

Suitable deodorant active ingredients are, for example, odor concealers such as the customary perfume constituents, odor absorbers, for example the sheet silicates described in the patent laid-open specification DE 40 09 347, of these, in particular montmorillonite, kaolinite, illite, beidelite, nontronite, saponite, hectorite, bentonite, smectite, or also, for example, zinc salts of ricinoleic acid. Antimicrobial agents are likewise suitable for being incorporated. Antimicrobial substances are, for example, 2,4,4′-trichloro-2′-hydroxydiphenyl ether (Irgasan), 1,6-di-(4-chlorophenylbiguanido)hexane(chlorhexidine), 3,4,4′-trichlorocarbonilide, quaternary ammonium compounds, clove oil, mint oil, thyme oil, triethyl citrate, farnesol (3,7,11-trimethyl-2,6,10-dodecatrien-1-ol), ethylhexyl glyceryl ether, polyglyceryl-3 caprylate (TEGO® Cosmo P813, Degussa), and the effective agents described in the patent laid-open specifications DE 198 55 934, DE 37 40 186, DE 39 38 140, DE 42 04 321, DE 42 29 707, DE 42 29 737, DE 42 38 081, DE 43 09 372, DE 43 24 219 and EP 666 732.

Antiperspirant active ingredients which may be used in the invention are astringents, for example basic aluminium chlorides such as aluminium chlorohydrate (“ACH”) and aluminium zirconium glycine salts (“ZAG”).

Insect repellents which may be used are, for example, N,N-diethyl-m-toluamide, 1,2-pentanediol or Insect Repellent 3535.

Self-tanning agents which can be used are, for example, dihydroxyacetone and erythrulose.

Preservatives which can be used are, for example, mixtures of one or more alkyl paraben esters with phenoxyethanol. The alkyl paraben esters may be methyl paraben, ethyl paraben, propyl paraben and/or butyl paraben. Instead of phenoxyethanol, it is also possible to use other alcohols, such as, for example, benzyl alcohol or ethanol. Moreover, it is also possible to use other customary preservatives such as, for example, sorbic acid or benzoic acid, salicylic acid, 2-bromo-2-nitropropane-1,3-diol, chloroacetamide, diazolidinylurea, DMDM hydantoin, iodopropynyl butylcarbamate, sodium hydroxymethylglycinates, methyl-isothiazoline, chloromethylisothiazoline, ethylhexylglycerol or caprylyl glycol.

Conditioning agents which can be used are, for example, organic quaternary compounds, such as cetrimonium chloride, dicetyldimonium chloride, behentrimonium chloride, distearyldimonium chloride, behentrimonium methosulphate, distearoylethyldimonium chloride, palmitamidopropyltrimonium chloride, guar hydroxypropyltrimonium chloride, hydroxypropylguar, hydroxypropyltrimonium chloride, or quaternium-80 or else amine derivatives such as, for example, aminopropyldimethicones or stearamidopropyldimethylamines.

Perfumes which can be used are natural or synthetic odorants or mixtures thereof. Natural odorants are extracts from flowers (lily, lavender, rose, jasmine, neroli, ylang ylang), stems and leaves (geranium, patchouli, petitgrain), fruits (anise, coriander, caraway, juniper), fruit peels (bergamot, lemon, orange), roots, (maize, angelica, celery, cardamon, costus, iris, thyme), needles and branches (spruce, fir, pine, dwarf-pine), resins and balsams (galbanum, elemi, benzoin, myrrh, olibanum, opoponax). Animal raw materials are also suitable, such as, for example, civet and castoreum. Typical synthetic odorant compounds are products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroycitronellal, lilial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include primarily the terpenes and balsams. It is possible to use mixtures of different odorants which together produce a pleasant scent note. Essential oils of low volatility, which are mostly used as aroma components, are also suitable as perfumes, e.g., sage oil, camomile oil, clove oil, Melissa oil, mint oil, cinnamon leaf oil, linden blossom oil, juniper berry oil, vetiver oil, olibanum oil, galbanum oil, labolanum oil and lavandin oil. It is also possible to use bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, fixolide NP, evernyl, iraldein gamma, phenylacetic acid, geranyl acetate, benzyl acetate, rose oxide, romillat, irotyl and floramat alone or in mixtures.

Dyes which can be used in the invention are the substances approved and suitable for cosmetic purposes, as are listed, for example, in the publication “Cosmetic Colourants” of the Dyes Commission of the German Research Society, Verlag Chemie, Weinheim, 1984, pp. 81 to 106. These dyes are usually used in concentrations of from 0.001 to 0.1% by weight, based on the total mixture.

Biogenic active ingredients are to be understood as meaning, for example, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, isoflavones, polyglutamic acid, creatine (and creatine derivatives), guanidine (and guanidine derivatives), pseudoceramides, essential oils, peptides, protein hydrolysates, plant extracts, bisabolol, allantoin, panthenol, phytantriol, idebenone, liquorice extract, glycyrrhizidine and idebenone, scleroglucan, β-glucan, santalbic acid and vitamin complexes. Examples of plant extracts are horsechestnut extract, camomile extract, rosemary extract, black and red currant extract, birch extract, rosehip extract, algae extract, green tea extract, aloe extract, ginseng extract, ginkgo extract, grapefruit extract, calendula extract, camphor, thyme extract, mangosteen extract, cystus extract, terminalia arjuna extract, oat extract, oregano extract, raspberry extract, strawberry extract, etc.

The biogenic active ingredients can also include the so-called barrier lipids, examples of which being ceramides, phytosphingosine and derivatives, sphingosine and derivatives, sphinganine and derivatives, pseudoceramides, phospholipids, lysophospholipids, cholesterol and derivatives, cholesteryl ester, free fatty acids, lanolin and derivatives, squalane, squalene and related substances.

Within the context of the invention, the biogenic active ingredients also include anti-acne, such as, for example, benzyl peroxide, phytosphingosine and derivatives, niacinamide hydroxybenzoate, nicotinaldehyde, retinol acid and derivatives, salicylic acid and derivatives, citronellic acid etc., and anti-cellulite, such as, for example, xanthine compounds such as caffeine, theophylline, theobromine and aminophylline, carnitine, camosine, salicyloyl phytosphingosine, phytosphingosines, santalbic acid etc., as well as antidandruff agents such as, for example, salicylic acid and derivatives, zinc pyrithione, selenium sulphide, sulphur, cyclopiroxolamine, bifonazole, climbazole, octopirox and actirox etc., as well as astringents, such as, for example, alcohol, aluminium derivatives, gallic acid, pyridoxine salicylate, zinc salts, such as, for example, zinc sulphate, acetate, chloride, lactate, zirconium chlorohydrates etc.

Bleaches such as kojic acid, arbutin, vitamin C and derivatives, hydroquinone, turmeric oil, creatinine, sphingolipids, niacinamide, etc. may likewise be included in the biogenic active ingredients.

Care additives which may be present are, for example, ethoxylated glycerol fatty acid esters, such as, for example, PEG-7 glycerol cocoate, or cationic polymers, such as, for example, polyquaternium-7 or polyglycerol esters.

Superfatting agents which can be used are substances such as, for example, lanolin and lecithin, and also polyethoxylated or acylated lanolin and lecithin derivatives, polyol fatty acid esters, monoglycerides and fatty acid alkanolamides, with the latter simultaneously serving as foam stabilizers.

Solvents which can be used are, for example, aliphatic alcohols such as ethanol, propanol or 1,3-propanediol, cyclic carbonates, such as ethylene carbonate, propylene carbonate, glycerol carbonate, esters of mono- or polycarboxylic acids such as ethyl acetate, ethyl lactate, dimethyl adipate and diethyl adipate, propylene glycol, dipropylene glycol, glycerol, glycerol carbonate or water.

The present invention is described by way of example in the examples listed below, without any intention to limit the invention, the scope of application of which arises from the entire description and the claims, to the embodiments specified in the examples.

### EXAMPLES

### Example 1

### Preparation of a Cationic Polysiloxane With Terminal Quaternary Functions

a) Equilibration of an SiH-functional polysiloxane
   16.4 g of phenyltris(dimethylsiloxy)silane, 232 g of decamethylpentasiloxane, 0.25 g of an acid catalyst were mixed in a 500 ml three-neck flask and stirred for 4 hours at 80° C. After cooling, 20 g of NaHCO₃ were added and the mixture was stirred at room temperature for 12 hours. After a filtration, a clear product was obtained.
b) Preparation of an epoxysiloxane
   300 g of the compound prepared according to 1a) and 9.3 g Allyl glycidyl ether were initially introduced together in a 500 ml three-neck flask and heated to 100° C. 15 ppm of a platinum catalyst were then added and the mixture was stirred for 2 hours. Following a subsequent reaction, a clear product with an epoxy value of 0.94% was obtained.
c) Conversion to the quaternary polysiloxane polymer
   36 g of 3-N,N-Dimethylaminopropyllaurylamide, 11 g of lactic acid and 100 g of isopropanol were stirred at room temperature in a 500 ml three-neck flask. 200 g of the compound prepared according to 1 b) were then added dropwise. The mixture was then stirred for 8 hours at 50° C. and distilled. A cloudy high-viscosity liquid was obtained, which is described by the following statistical formula:

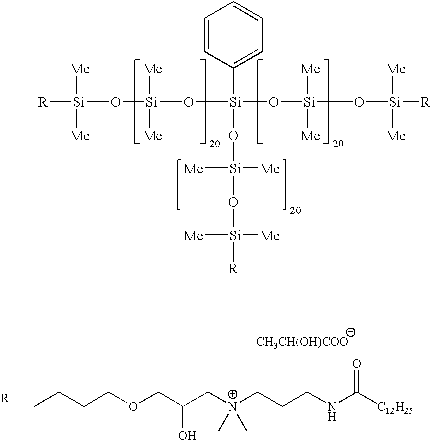

A person skilled in the art will appreciate that the formula given above represents an idealized structural formula. Linear and more highly branched structures are additionally present in the product.

### Example 2

### Preparation of a Linear Cationic Polysiloxane With Two Terminal Quaternary Functions

a) Equilibration of an SiH-functional polysiloxane (α,ωN=60)
   10 g of 1,1,3,3-tetramethyldisiloxane, 335 g of decamethylpentasiloxane, 0.35 g of an acidic catalyst were mixed in a 500 ml three-neck flask and stirred for 4 hours at 80° C. After cooling, 6 g of NaHCO₃ were added and the mixture was stirred at room temperature for 12 hours. After a filtration, a clear product with an SiH value of 0.04% was obtained.
b) Preparation of an epoxysiloxane (α,ωN=60)
   250 g of the compound prepared according to 2a and 8 g of allyl glycidyl ether were initially introduced together in a 500 ml three-neck flask and heated to 100° C. 15 ppm of a platinum catalyst were then added and the mixture was stirred for 3 hours. The reaction mixture was then freed from volatile constituents in an oil pump vacuum at 120° C. for 2 hours. A clear product with an epoxy value of 0.65% was obtained.
c) Conversion to the quaternary polysiloxane polymer (α,ωN=60)
   27 g of 3-N,N-dimethylaminopropyllaurylamide, 5.5 g of acetic acid and 130 g of isopropanol were stirred at room temperature in a 500 ml three-neck flask. 200 g of the compound prepared according to 2b were then added dropwise. The mixture was then stirred for 8 hours at 50° C. and finally the volatile fractions were removed from the reaction mixture in the oil pump vacuum at 100° C. A cloudy high-viscosity liquid was obtained, which is described by the following statistical formula:

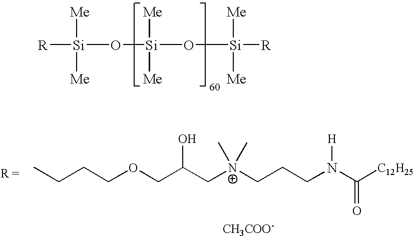

### Example 3

### Applications-Related Properties of the Cationic Polysiloxanes With Quaternary Functions

Preparation and testing of hair treatment compositions using the compounds of example 1 and example 2.

For the applications-related assessment, hair tresses pre-damaged in a standardized way by a bleaching treatment were used. For this, standard hairdressing products were used. The damage to the hair tresses is described in detail in DE 103 27 871.

Test Formulations and Pre-Treatment of the Hair Tresses:

For the applications-related assessment, the compounds of example 1 and example 2 were used in a simple cosmetic formulation.

The application properties upon use in hair rinses were investigated in the following formulations (Table 1):

TABLE 1
Hair rinse formulations for testing the hair conditioning properties.

| | Formulation | | |
|---|---|---|---|
| | examples | | |
| | 0a | 1a | 2a |
| TEGINACID ® C, Evonik Goldschmidt GmbH | 0.5% | 0.5% | 0.5% |
| (INCI: Ceteareth-25) | | | |
| TEGO ® Alkanol 16, Evonik Goldschmidt | 4% | 4% | 4% |
| GmbH | | | |
| (INCI: Cetyl Alcohol)) | | | |
| VARISOFT ® 300, 30% strength, Evonik | 3.3% | 3.3% | 3.3% |
| Goldschmidt GmbH | | | |
| (INCI: Cetrimonium Chloride) | | | |
| Water, demineralized | ad 100.0 | | |
| Citric acid | ad pH 4.0 ± 0.3 | | |
| Compound of example 1 | — | 0.5% | — |
| Compound of example 2 | — | — | 0.5% |

The composition of the test formulations was deliberately chosen to be simple to avoid the influencing of the test results by (normally present) formulation constituents.

Formulations according to the invention can comprise further ingredients besides these specific ingredients and/or instead of the specific ingredients. In particular, the combination with further ingredients can lead to a synergistic improvement for the described effects.

The hair is pre-treated using a shampoo formulation (Table 2) which does not contain a conditioner.

TABLE 2
Shampoo formulation for the pre-treatment of the hair tresses.

| | |
|---|---|
| Texapon NSO ®, 28% strength, Cognis | 42.9% |
| (INCI: Sodium Laureth Sulfate) | |
| NaCl | 3% |
| Water, demineralized | ad 100.0 |

Standardized Treatment of Pre-Damaged Hair Tresses With Conditioning Samples:

The predamaged hair tresses, as described above, were washed with the shampoo formulation in Table 2. For this, the hair tresses were wetted under running warm water. The excess water was gently squeezed out by hand, then the shampoo was applied and gently worked into the hair for 1 min (0.5 ml/2 g hair tress). The hair tress was rinsed under running warm water for 30 s. This procedure was repeated once more except that rinsing was carried out at the end for 1 min.

Directly after washing, the hair tresses were then conditioned with the hair rinse formulations in Table 1.

For this, the rinse was applied and gently worked into the hair (0.5 ml/2 g hair tress). After a residence time of 1 min, the hair was rinsed for 1 min.

The hair tresses conditioned with the hair rinse formulations were then dried in the air at 50% atmospheric humidity and 25° C. for at least 12 h prior to damaging the hair tresses with the smoothing iron and the subsequent DSC measurements (Differential Scanning Calorimetry).

Damaging the Hair Tresses:

For the heat damage of the hair tresses, a Babyliss Pro Tourmaline Pulse smoothing iron was used.

Prior to the heat treatment of the hair tresses, the smoothing iron was preheated to the highest setting (setting 5, corresponds ca. to a temperature of 190° C.).

The hair tresses were then treated with the smoothing iron three times for 10 seconds in each case.

Determination of the Hair Damage Due to Heat Treatment With Smoothing Iron:

The hair damaged with the smoothing iron was then measured using differential scanning calorimetry in order to determine the degree of damage to the hair. Differential scanning colorimetry is a standard method for determining the degree of damage to hair. Details are described in the article by F. J. Wortmann “Investigations of cosmetically treated human hair by differential scanning calorimetry in water” (J. Cosmet. Sci., 53, 219-228, 2002). In this measurement method, lower temperatures are ascertained with increasing hair damage.

As reference value, a hair tress treated with formulation 0a which was not treated with the smoothing iron was measured using differential scanning calorimetry (DSC).

DSC Measurement Results:

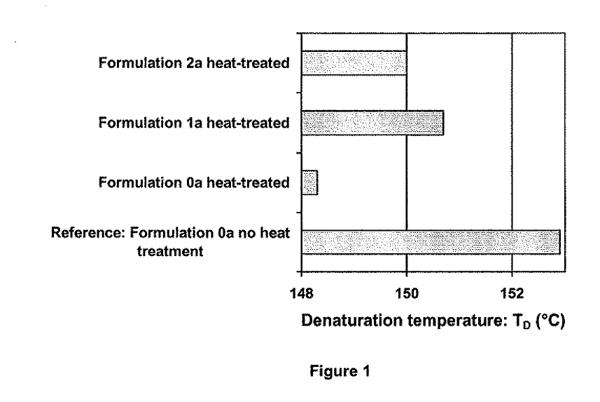

FIG. 1 summarizes the DSC measurement results obtained. The reference (hair tress conditioned with formulation 0a, no subsequent heat treatment) had the highest denaturation temperature and therefore the lowest damage to the hair. The hair tress which was conditioned with formulation 0a and then treated with the smoothing iron had the lowest denaturation temperature and the greatest damage to the hair was present. When using the compounds of example 1 and example 2 in the formulations 1a and 2a, significantly higher denaturation temperatures were measured. Consequently, damage to the hair was reduced through the use of the compounds of example 1 and 2. The effect was more marked when using example 1 than when using example 2.

### FORMULATION EXAMPLES

These formulation examples show that polysiloxanes with terminal quaternary ammonium groups can be used in a large number of cosmetic formulations to protect the hair against heat damage.

### Formulation Example 1

### Clear Shampoo

| | |
|---|---|
| TEXAPON ® NSO, Cognis, 28% strength (INCI: | 32.00% |
| Sodium Laureth Sulfate) | |
| Compound of example 1 | 0.50% |
| Perfume | 0.5% |
| Water | 57.5% |
| TEGO ® Betain F 50, Evonik Goldschmidt GmbH, 38% | 8.0% |
| strength (INCI: Cocamidopropyl Betaine) | |
| ANTIL ® 171 Evonik Goldschmidt GmbH (INCI: PEG- | 1.0% |
| 18 Glyceryl Oleate/Cocoate) | |
| NaCl | 0.0% |
| Preservative | q.s. |

### Formulation Example 2

### Clear Conditioning Shampoo

| | |
|---|---|
| TEXAPON ® NSO, Cognis, 28% strength (INCI: | 32.0% |
| Sodium Laureth Sulfate) | |
| Compound of example 1 | 1.0% |
| Perfume | 0.5% |
| Water | 57.5 |
| TEGO ® Cosmo C 100, Evonik Goldschmidt GmbH, | 1.0% |
| (INCI: Creatine) | |
| Jaguar C-162, Rhodia (INCI: Hydroxypropyl Guar | 0.20 |
| Hydroxypropyltrimonium Chloride) | |
| TEGO ® Betain F 50, Evonik Goldschmidt GmbH, 38% | 8.00% |
| strength (INCI: Cocamidopropyl Betaine) | |
| NaCl | 0.50% |
| Preservative | q.s. |

### Formulation Example 3

### Clear Conditioning Shampoo

| | |
|---|---|
| TEXAPON ® NSO, Cognis, 28% strength (INCI: | 32.00% |
| Sodium Laureth Sulfate) | |
| ANTIL ® 200, Evonik Goldschmidt GmbH (INCI: PEG- | 2.00% |
| 200 Hydrogenated Glyceryl Palmate; PEG-7 Glyceryl | |
| Cocoate) | |
| Compound of example 1 | 1.00% |
| Perfume | 0.25% |
| Water | 56.25 |
| Polymer JR 400, Amerchol (INCI: Polyquaternium-10) | 0.20 |
| TEGO ® Betain F 50, Evonik Goldschmidt GmbH, 38% | 8.00% |
| strength (INCI: Cocamidopropyl Betaine) | |
| NaCl | 0.30% |
| Preservative | q.s. |

### Formulation Example 4

### Clear Conditioning Shampoo

| | |
|---|---|
| TEXAPON ® NSO, Cognis, 28% strength (INCI: | 32.00% |
| Sodium Laureth Sulfate) | |
| ANTIL ® 200, Evonik Goldschmidt GmbH (INCI: PEG- | 2.00% |
| 200 Hydrogenated Glyceryl Palmate; PEG-7 Glyceryl | |
| Cocoate) | |
| ABIL ® Quat 3272, Evonik Goldschmidt GmbH (INCI: | 0.75% |
| Quaternium-80) | |
| Compound of example 1 | 0.50% |
| Perfume | 0.25% |
| Water | 56.00 |
| Polymer JR 400, Amerchol (INCI: Polyquaternium-10) | 0.20 |
| TEGO ® Betain F 50, Evonik Goldschmidt GmbH, 38% | 8.00% |
| strength (INCI: Cocamidopropyl Betaine) | |
| NaCl | 0.30% |
| Preservative | q.s. |

### Formulation Example 5

### Clear Conditioning Shampoo

| | |
|---|---|
| TEXAPON ® NSO, Cognis, 28% strength (INCI: | 32.00% |
| Sodium Laureth Sulfate) | |
| ANTIL ® 200, Evonik Goldschmidt GmbH (INCI: PEG- | 2.00% |
| 200 Hydrogenated Glyceryl Palmate; PEG-7 Glyceryl | |
| Cocoate) | |
| ABIL ® B 8832, Evonik Goldschmidt GmbH (INCI: Bis- | 1.00% |
| PEG/PPG-20/20 Dimethicone) | |
| Compound of example 1 | 0.50% |
| Perfume | 0.25% |
| Water | 55.75 |
| Polymer JR 400, Amerchol (INCI: Polyquaternium-10) | 0.20 |
| TEGO ® Betain F 50, Evonik Goldschmidt GmbH, 38% | 8.00% |
| strength (INCI: Cocamidopropyl Betaine) | |
| NaCl | 0.30% |
| Preservative | q.s. |

### Formulation Example 6

### Clear Conditioning Shampoo

| | |
|---|---|
| TEXAPON ® NSO, Cognis, 28% strength (INCI: | 32.00% |
| Sodium Laureth Sulfate) | |
| VARISOFT ® PATC, Evonik Goldschmidt GmbH(INCI: | 1.50% |
| Palmitamidopropyltrimonium Chloride) | |
| REWODERM ® LI S 80, Evonik Goldschmidt GmbH | 2.00% |
| (INCI: PEG-200 Hydrogenated Glyceryl Palmate; PEG- | |
| 7 Glyceryl Cocoate) | |
| Compound of example 1 | 0.50% |
| Perfume | 0.25% |
| Water | 54.05 |
| TEGO ® Cosmo C 100, Evonik Goldschmidt GmbH, | 1.00% |
| (INCI: Creatine) | |
| Jaguar C-162, Rhodia (INCI: Hydroxypropyl Guar | 0.20 |
| Hydroxypropyltrimonium Chloride) | |
| TEGO ® Betain F 50, Evonik Goldschmidt GmbH, 38% | 8.00% |
| strength (INCI: Cocamidopropyl Betaine) | |
| NaCl | 0.50% |
| Preservative | q.s. |

### Formulation Example 7

### Clear Conditioning Shampoo

| | |
|---|---|
| TEXAPON ® NSO, Cognis, 28% strength (INCI: | 32.00% |
| Sodium Laureth Sulfate) | |
| REWODERM ® LI S 80, Evonik Goldschmidt GmbH | 2.00% |
| (INCI: PEG-200 Hydrogenated Glyceryl Palmate; PEG- | |
| 7 Glyceryl Cocoate) | |
| Compound of example 1 | 0.50% |
| Perfume | 0.25% |
| Water | 55.55 |
| TEGO ® Cosmo C 100, Evonik Goldschmidt GmbH, | 1.00% |
| (INCI: Creatine) | |
| Jaguar C-162, Rhodia (INCI: Hydroxypropyl Guar | 0.20 |
| Hydroxypropyltrimonium Chloride) | |
| TEGO ® Betain F 50, Evonik Goldschmidt GmbH, 38% | 8.00% |
| strength (INCI: Cocamidopropyl Betaine) | |
| NaCl | 0.50% |
| Preservative | q.s. |

### Formulation Example 8

### Pearlized Shampoo

| | |
|---|---|
| TEXAPON ® NSO, Cognis, 28% strength (INCI: | 32.00% |
| Sodium Laureth Sulfate) | |
| Compound of example 1 | 0.50% |
| Perfume | 0.25% |
| Water | 55.25 |
| TEGO ® Betain F 50, Evonik Goldschmidt GmbH, 38% | 8.00% |
| strength (INCI: Cocamidopropyl Betaine) | |
| TEGO ® Pearl N 300 Evonik Goldschmidt GmbH (INCI: | 2.00% |
| Glycol Distearate; Laureth-4; Cocamidopropyl Betaine) | |
| ANTIL ® 171 Evonik Goldschmidt GmbH (INCI: PEG- | 1.50% |
| 18 Glyceryl Oleate/Cocoate) | |
| NaCl | 0.50% |
| Preservative | q.s. |

### Formulation Example 9

### 2 in 1 Shampoo

| | |
|---|---|
| TEXAPON ® NSO, Cognis, 28% strength (INCI: | 32.00% |
| Sodium Laureth Sulfate) | |
| VARISOFT ® PATC, Evonik Goldschmidt GmbH(INCI: | 1.50% |
| Palmitamidopropyltrimonium Chloride) | |
| REWODERM ® LI S 80, Evonik Goldschmidt GmbH | 2.00% |
| (INCI: PEG-200 Hydrogenated Glyceryl Palmate; PEG- | |
| 7 Glyceryl Cocoate) | |
| Compound of example 1 | 0.50% |
| Perfume | 0.25% |
| Water | 54.05 |
| TEGO ® Cosmo C 100, Evonik Goldschmidt GmbH, | 1.00% |
| (INCI: Creatine) | |
| Jaguar C-162, Rhodia (INCI: Hydroxypropyl Guar | 0.20 |
| Hydroxypropyltrimonium Chloride) | |
| TEGO ® Betain F 50, Evonik Goldschmidt GmbH, 38% | 8.00% |
| strength (INCI: Cocamidopropyl Betaine) | |
| NaCl | 0.50% |
| Preservative | q.s. |

### Formulation Example 10

### Rinse-Off Conditioner

| | |
|---|---|
| Water | 90.50% |
| VARISOFT ® BT 85, Evonik Goldschmidt GmbH | 3.00% |
| (INCI: Behentrimonium Chloride) | |
| Compound of example 1 | 1.50% |
| TEGO ® Alkanol 1618, Evonik Goldschmidt GmbH | 5.00 |
| (INCI: Cetearyl Alcohol) | |
| Preservative, Perfume | q.s. |

### Formulation Example 11

### Rinse-Off Conditioner

| | |
|---|---|
| Water | 90.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| Compound of example 1 | 0.80% |
| TEGO ® Alkanol 1618, Evonik Goldschmidt GmbH | 5.00 |
| (INCI: Cetearyl Alcohol) | |
| Preservative, Perfume | q.s. |

### Formulation Example 12

### Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt (GmbH | 2.00% |
| (INCI: Behentrimonium Chloride) | |
| ABIL ® Quat 3272, Evonik Goldschmidt GmbH (INCI: | 1.00% |
| Quaternium-80) | |
| Compound of example 1 | 0.80% |
| TEGO ® Alkanol 1618, Evonik Goldschmidt GmbH | 5.00 |
| (INCI: Cetearyl Alcohol) | |
| Preservative, Perfume | q.s. |

### Formulation Example 13

### Rinse-Off Conditioner

| | |
|---|---|
| TEGINACID ® C, Evonik Goldschmidt GmbH (INCI: | 0.50% |
| Ceteareth-25) | |
| TEGO ® Alkanol 16, Evonik Goldschmidt GmbH (INCI: | 2.00% |
| Cetyl Alcohol) | |
| TEGO ® Amid S 18, Evonik Goldschmidt GmbH (INCI: | 1.00% |
| Stearamidopropyl Dimethylamine) | |
| Compound of example 1 | 1.50% |
| Propylene Glycol | 2.00% |
| Citric Acid Monohydrate | 0.30% |
| Water | 92.70% |
| Preservative, Perfume | q.s. |

### Formulation Example 14

### Rinse-Off Conditioner

| | |
|---|---|
| TEGINACID ® C, Evonik Goldschmidt GmbH (INCI: | 0.50% |
| Ceteareth-25) | |
| TEGO ® Alkanol 16, Evonik Goldschmidt GmbH (INCI: | 5.00% |
| Cetyl Alcohol) | |
| TEGOSOFT ® DEC, Evonik Goldschmidt GmbH (INCI: | 1.00% |
| Diethylhexyl Carbonate) | |
| Compound of example 1 | 1.50% |
| Water | 89.20% |
| TEGO ® Cosmo C 100 Evonik Goldschmidt GmbH | 0.50% |
| (INCI: Creatine) | |
| Propylene Glycol | 2.00% |
| Citric Acid Monohydrate | 0.30% |
| Preservative, Perfume | q.s. |

### Formulation Example 15

### Leave-In Conditioner Spray

| | |
|---|---|
| Lactic Acid, 80% | 0.40% |
| Water | 95.30% |
| TEGO ® Amid S 18, Evonik Goldschmidt GmbH (INCI: | 1.20% |
| Stearamidopropyl Dimethylamine) | |
| TEGIN ® G 1100 Pellets, Evonik Goldschmidt GmbH | 0.60% |
| (INCI: Glycol Distearate) | |
| TEGO ® Care PS, Evonik Goldschmidt GmbH (INCI: | 1.20% |
| Methyl Glucose Sesquistearate) | |
| TEGOSOFT ® DEC, Evonik Goldschmidt GmbH (INCI: | 0.30% |
| Diethylhexyl Carbonate) | |
| Compound of example 1 | 1.00% |
| Preservative, Perfume | q.s. |

### Formulation Example 16

### Leave-In Conditioner Spray

| | |
|---|---|
| TAGAT ® CH-40, Evonik Goldschmidt GmbH (INCI: | 2.00% |
| PEG-40 Hydrogenated Castor Oil) | |
| Ceramide VI, Evonik Goldschmidt GmbH (INCI: | 0.05% |
| Ceramide 6 II) | |
| Perfume | 0.20% |
| Water | 90.95% |
| Compound of example 1 | 0.50% |
| LACTIL ® Evonik Goldschmidt GmbH (INCI: Sodium | 2.00% |
| Lactate; Sodium PCA; Glycine; Fructose; Urea; | |
| Niacinamide; Inositol; Sodium benzoate; Lactic Acid) | |
| TEGO ® Betain F 50 Evonik Goldschmidt GmbH 38% | 2.30% |
| (INCI: Cocamidopropyl Betaine) | |
| Citric Acid (10% in water) | 2.00% |

### Formulation Example 17

### Leave-In Conditioner Foam

| | |
|---|---|
| Compound of example 1 | 0.50% |
| TAGAT ® CH-40, Evonik Goldschmidt GmbH (INCI: | 0.50% |
| PEG-40 Hydrogenated Castor Oil) | |
| Perfume | 0.30% |
| TEGO ® Betain 810, Evonik Goldschmidt GmbH (INCI: | 2.00% |
| Capryl/Capramidopropyl Betaine) | |
| Water | 94.00% |
| TEGO ® Cosmo C 100, Evonik Goldschmidt GmbH | 0.50% |
| (INCI: Creatine) | |
| TEGOCEL ® HPM 50, Evonik Goldschmidt GmbH | 0.30% |
| (INCI: Hydroxypropyl Methylcellulose) | |
| VARISOFT ® 300, Evonik Goldschmidt GmbH (INCI: | 1.30% |
| Cetrimonium Chloride) | |
| LACTIL ® Evonik Goldschmidt GmbH (INCI: Sodium | 0.50% |
| Lactate; Sodium PCA; Glycine; Fructose; Urea; | |
| Niacinamide; Inositol; Sodium benzoate; Lactic Acid) | |
| Citric Acid, 30% | 0.10% |
| Preservative | q.s. |

### Formulation Example 18

### Strong Hold Styling Gel

| | |
|---|---|
| TEGO ® Carbomer 141, Evonik Goldschmidt GmbH | 1.20% |
| (INCI: Carbomer) | |
| Water | 67.00% |
| NaOH, 25% | 2.70% |
| PVP/VA W-735, ISP (INCI: PVP/VA Copolymer | 16.00% |
| Compound of example 1 | 0.50% |
| Alcohol Denat. | 10.30% |
| TAGAT ® O 2 V, Evonik Goldschmidt GmbH (INCI: | 2.00% |
| PEG-20 Glyceryl Oleate) | |
| Perfume | 0.30% |
| ABIL ® B 88183, Evonik Goldschmidt GmbH (INCI: | 0.30% |
| PEG/PPG-20/6 Dimethicone) | |
| Preservative | q.s. |

### Formulation Example 19

### Foamy Body Care Composition

| | |
|---|---|
| TEXAPON ® NSO, Cognis, 28% strength (INCI: | 14.30% |
| Sodium Laureth Sulfate) | |
| Perfume | 0.30% |
| Compound of example 1 | 0.50% |
| REWOTERIC ® AM C, Evonik Goldschmidt GmbH, | 8.00% |
| 32% strength (INCI: Sodium Cocoamphoacetate) | |
| Water | 74.90% |
| TEGOCEL ® HPM 50, Evonik Goldschmidt GmbH | 0.50% |
| (INCI: Hydroxypropyl Methylcellulose) | |
| LACTIL ®, Evonik Goldschmidt GmbH (INCI: Sodium | 1.00% |
| Lactate; Sodium PCA; Glycine; Fructose; Urea; | |
| Niacinamide; Inositol; Sodium benzoate; Lactic Acid) | |
| Citric Acid Monohydrate | 0.50% |

### Formulation Example 20

### Body Care Composition

| | |
|---|---|
| TEXAPON ® NSO, Cognis, 28% strength (INCI: | 30.00% |
| Sodium Laureth Sulfate) | |
| TEGOSOFT ® PC 31, Evonik Goldschmidt GmbH | 0.50% |
| (INCI: Polyglyceryl-3 Caprate) | |
| Compound of example 1 | 0.50% |
| Perfume | 0.30% |
| Water | 53.90% |
| TEGOCEL ® HPM 4000, Evonik Goldschmidt GmbH | 0.30% |
| (INCI: Hydroxypropyl Methylcellulose) | |
| REWOTERIC ® AM C, Evonik Goldschmidt GmbH, | 10.00% |
| 32% strength (INCI: Sodium Cocoamphoacetate) | |
| Citric Acid Monohydrate | 0.50% |
| REWODERM ® LI S 80, Evonik Goldschmidt GmbH | 2.00% |
| (INCI: PEG-200 Hydrogenated Glyceryl Palmate; PEG- | |
| 7 Glyceryl Cocoate) | |
| TEGO ® Pearl N 300, Evonik Goldschmidt GmbH | 2.00% |
| (INCI: Glycol Distearate; Laureth-4; Cocamidopropyl | |
| Betaine) | |

### Formulation Example 21

### Foamy Body Care Composition

| | |
|---|---|
| TEXAPON ® NSO, Cognis, 28% strength (INCI: | 14.30% |
| Sodium Laureth Sulfate) | |
| Perfume | 0.30% |
| Compound of example 1 | 0.50% |
| REWOTERIC ® AM C, Evonik Goldschmidt GmbH, | 8.00% |
| 32% strength (INCI: Sodium Cocoamphoacetate) | |
| Water | 75.10% |
| Polyquaternium-7 | 0.30 |
| LACTIL ®, Evonik Goldschmidt GmbH (INCI: Sodium | 1.00% |
| Lactate; Sodium PCA; Glycine; Fructose; Urea; | |
| Niacinamide; Inositol; Sodium beuzoate; Lactic Acid) | |
| Citric Acid Monohydrate | 0.50% |

### Formulation Example 22

### Mild Foam Bath

| | |
|---|---|
| TEXAPON ® NSO, Cognis, 28% strength (INCI: | 27.00% |
| Sodium Laureth Sulfate) | |
| REWOPOL ® SB FA 30, Evonik Goldschmidt GmbH, | 12.00% |
| 40% strength (INCI: Disodium Laureth Sulfosuccinate) | |
| TEGOSOFT ® LSE 65 K SOFT, Evonik Goldschmidt | 2.00% |
| GmbH (INCI: Sucrose Cocoate) | |
| Water | 39.00% |
| REWOTERIC ® AM C, Evonik Goldschmidt GmbH, | 13.00% |
| 32% strength (INCI: Sodium Cocoamphoacetate) | |
| Compound of example 1 | 0.50% |
| Citric Acid (30% in water) | 3.00% |
| ANTIL ® 171 Evonik Goldschmidt GmbH (INCI: PEG- | 1.50% |
| 18 Glyceryl Oleate/Cocoate) | |
| TEGO ® Pearl N 300 Evonik Goldschmidt GmbH (INCI: | 2.00% |
| Glycol Distearate; Laureth-4; Cocamidopropyl Betaine) | |

### Formulation Example 23

### Foamy Body Care Composition

| | |
|---|---|
| TEGOCEL ® HPM 50, Evonik Goldschmidt GmbH | 0.50% |
| (INCI: Hydroxypropyl Methylcellulose) | |
| Water | 80.10% |
| Perfume | 0.20% |
| Compound of example 1 | 0.50% |
| TEGOSOFT ® GC, Evonik Goldschmidt GmbH, (INCI: | 1.30% |
| PEG-7 Glyceryl Cocoate) | |
| TEGO ® Betain 810, Evonik Goldschmidt GmbH (INCI: | 16.90% |
| Capryl/Capramidopropyl Betaine) | |
| LACTIL ®, Evonik Goldschmidt GmbH (INCI: Sodium | 0.50% |
| Lactate; Sodium PCA; Glycine; Fructose; Urea; | |
| Niacinamide; Inositol; Sodium benzoate; Lactic Acid) | |
| Preservative | q.s. |

### Formulation Example 24

### Clear Shampoo

| | |
|---|---|
| TEXAPON ® NSO, Cognis, 28% strength (INCI: | 32.00% |
| Sodium Laureth Sulfate) | |
| Compound of example 1 | 0.50% |
| Perfume | 0.25% |
| Water | 56.05% |
| TEGO ® Betain F 50, Evonik Goldschmidt GmbH, 38% | 8.00% |
| strength (INCI: Cocamidopropyl Betaine) | |
| ANTIL ® 171 Evonik Goldschmidt GmbH (INCI: PEG- | 2.50% |
| 18 Glyceryl Oleate/Cocoate) | |
| NaCl | 0.70% |
| Preservative | q.s. |

### Formulation Example 25

### Clear Shampoo

| | |
|---|---|
| TEXAPON ® NSO, Cognis, 28% strength (INCI: | 32.00% |
| Sodium Laureth Sulfate) | |
| Compound of example 1 | 0.50% |
| Perfume | 0.25% |
| Water | 55.35% |
| REWOTERIC ® AMC, Evonik Goldschmidt GmbH, | 9.40% |
| (INCI: Sodium Cocoamphoacetate) | |
| ANTIL ® 171 Evonik Goldschmidt GmbH (INCI: PEG- | 2.50% |
| 18 Glyceryl Oleate/Cocoate) | |
| Preservative | q.s. |

### Formulation Example 25

### Clear Shampoo

| | |
|---|---|
| TEXAPON ® NSO, Cognis, 28% strength (INCI: | 17.90% |
| Sodium Laureth Sulfate) | |
| Compound of example 1 | 0.50% |
| Perfume | 0.25% |
| Water | 62.50% |
| TEGO ® Betain F 50, Evonik Goldschmidt GmbH, 38% | 6.60% |
| strength (INCI: Cocamidopropyl Betaine) | |
| REWOPOL ® SB FA 30, Evonik Goldschmidt GmbH, | 6.25% |
| (INCI: Disodium Laureth Sulfosuccinate) | |
| ANTIL ® 171 Evonik Goldschmidt GmbH (INCI: PEG- | 5.00% |
| 18 Glyceryl Oleate/Cocoate) | |
| NaCl | 1.00% |
| Preservative | q.s. |

### Formulation Example 26

### Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| ABIL ® OSW 5, Evonik Goldschmidt GmbH (INCI: | 1.00% |
| Cyclopentasiloxane; Dimethiconol) | |
| Compound of example 1 | 0.80% |
| TEGO ® Alkanol 1618, Evonik Goldschmidt GmbH | 5.00 |
| (INCI: Cetearyl Alcohol) | |
| Preservative, Perfume | q.s. |

### Formulation Example 27

### Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| ABIL ® Soft AF 100, Evonik Goldschmidt GmbH (INCI: | 1.00% |
| Methoxy PEG/PPG-7/3 Aminopropyl Dimethicone) | |
| Compound of example 1 | 0.80% |
| TEGO ® Alkanol 1618, Evonik Goldschmidt GmbH | 5.00 |
| (INCI: Cetearyl Alcohol) | |
| Preservative, Perfume | q.s. |

### Formulation Example 28

### Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| SF 1708, Momentive (INCI: Amodimethicone) | 1.00% |
| Compound of example 1 | 0.80% |
| TEGO ® Alkanol 1618, Evonik Goldschmidt GmbH | 5.00 |
| (INCI: Cetearyl Alcohol) | |
| Preservative, Perfume | q.s. |

### Formulation Example 29

### Conditioning Shampoo

| | |
|---|---|
| TEXAPON ® NSO, Cognis, 28% strength (INCI: | 27.00% |
| Sodium Laureth Sulfate) | |
| Plantacare 818 UP, Cognis 51.4% strength (INCI: Coco | 5.00% |
| Glucoside) | |
| T-Quat | 1.50% |
| Perfume | 0.25% |
| Water | 56.55 |
| TEGO ® Cosmo C 100, Evonik Goldschmidt GmbH, | 1.00% |
| (INCI: Creatine) | |
| Jaguar C-162, Rhodia (INCI: Hydroxypropyl Guar | 0.20 |
| Hydroxypropyltrimonium Chloride) | |
| TEGO ® Betain F 50, Evonik Goldschmidt GmbH, 38% | 8.00% |
| strength (INCI: Cocamidopropyl Betaine) | |
| NaCl | 0.50% |
| Preservative | q.s. |

### Formulation Example 30

### Conditioning Shampoo

| | |
|---|---|
| Plantacare 818 UP, Cognis 51.4% strength (INCI: Coco | 18.00% |
| Glucoside) | |
| T-Quat | 1.50% |
| Perfume | 0.25% |
| Water | 70.55% |
| TEGO ® Cosmo C 100, Evonik Goldschmidt GmbH, | 1.00% |
| (INCI: Creatine) | |
| Jaguar C-162, Rhodia (INCI: Hydroxypropyl Guar | 0.20 |
| Hydroxypropyltrimonium Chloride) | |
| TEGO ® Betain F 50, Evonik Goldschmidt GmbH, 38% | 8.00% |
| strength (INCI: Cocamidopropyl Betaine) | |
| NaCl | 0.50% |
| Preservative | q.s. |

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A method for protecting animal or human hair against heat damage comprising:
applying a formulation including a polysiloxane containing at least one quaternary ammonium group to hair.

2. The method of claim 1 wherein the at least one quaternary ammonium group is terminally bonded to the polysiloxane.

3. The method of claim 1 wherein exactly two quaternary ammonium groups are present and the two quaternary ammonium groups are terminally bonded to the polysiloxane.

4. The method of claim 1 wherein the polysiloxane is a compound of formula I
[M′Dₙ]₃T Formula I
where
M′=XSiY₂O_{1/2},
D=SiY₂O_{2/2},
T=SiZO_{3/2},
X=identical or different organic radicals which carry quaternary ammonium groups,
Y=identical or different radicals from the group alkyl, aryl, or alkaryl having 1 to 30 carbon atoms,
Z=identical or different radicals from the group alkyl, aryl or alkaryl having 1 to 30 carbon atoms, and
n=2 to 200.

5. The method of claim 4 wherein X is a radical having the structure —R1-R2, where
R1 are identical or different divalent radicals selected from the group 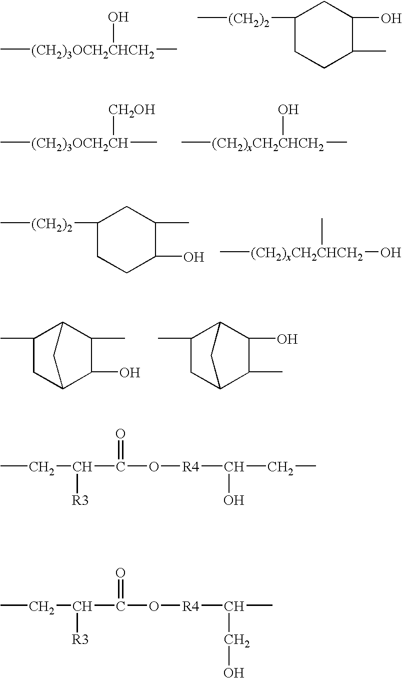
R2 is selected from the group consisting of 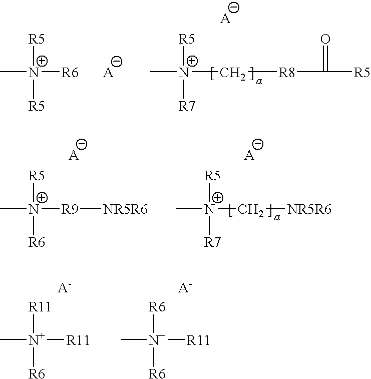
R3 are identical or different radicals from the group hydrogen or alkyl having 1 to 6 carbon atoms,
R4 are identical or different divalent hydrocarbon radicals which optionally comprise ether functions,
R5, R6, R7, in each case independently of one another, are hydrogen or alkyl radicals having 1 to 30 carbon atoms,
R8 are identical or different radicals from the group —O—; —NR10,
R9 are identical or different optionally branched divalent hydrocarbon radicals,
R10 are identical or different radicals from the group hydrogen or alkyl having 1 to 6 carbon atoms,
R11 are identical or different radicals of the general formula 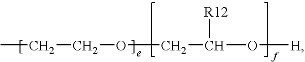
R12 are identical or different alkyl, aryl, or alkaryl radicals having 1 to 30 carbon atoms which optionally comprise ether functions,
e is 0 to 20,
f is 0 to 20,
e+f is >=1,
x is 2 to 18,
a is 2 to 18,
and
A⁻ are identical or different counterions to the positive charges on the quaternary ammonium groups, selected from inorganic or organic anions of the acids HA.

6. The method of claim 1 wherein the formulation is a cosmetic formulation.

7. The method of claim 1 wherein the formulation is a cleaning and care formulation.

8. A method for protecting animal or human hair against heat damage comprising:
providing a formulation including a polysiloxane containing at least one quaternary ammonium group; and
applying said formulation to hair.

9. The method of claim 8 wherein the at least one quaternary ammonium group is terminally bonded to the polysiloxane.

10. The method of claim 8 wherein exactly two quaternary ammonium groups are present and the two quaternary ammonium groups are terminally bonded to the polysiloxane.

11. The method of claim 8 wherein the polysiloxane is a compound of formula I
[M′Dₙ]₃T Formula I
where
M′=XSiY₂O_{1/2},
D=SiY₂O_{2/2},
T=SiZO_{3/2},
X=identical or different organic radicals which carry quaternary ammonium groups,
Y=identical or different radicals from the group alkyl, aryl, or alkaryl having 1 to 30 carbon atoms,
Z=identical or different radicals from the group alkyl, aryl or alkaryl having 1 to 30 carbon atoms, and
n=2 to 200.

12. The method of claim 11 wherein X is a radical having the structure —R1-R2, where
R1 are identical or different divalent radicals selected from the group 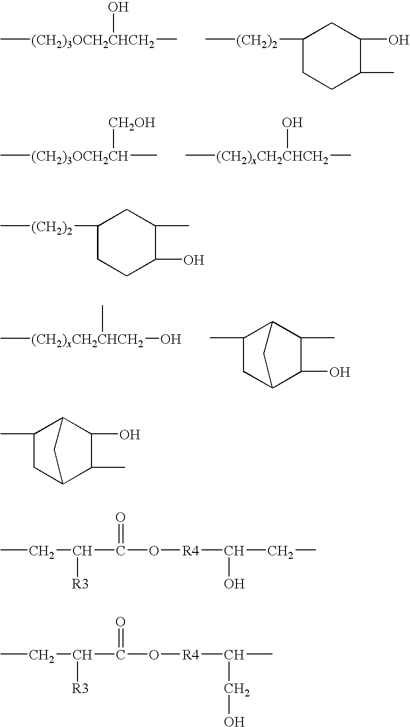
R2 is selected from the group consisting of 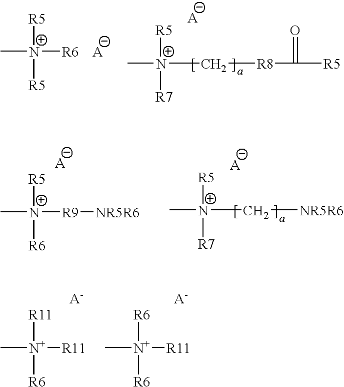
R3 are identical or different radicals from the group hydrogen or alkyl having 1 to 6 carbon atoms,
R4 are identical or different divalent hydrocarbon radicals which optionally comprise ether functions,
R5, R6, R7, in each case independently of one another, are hydrogen or alkyl radicals having 1 to 30 carbon atoms,
R8 are identical or different radicals from the group —O—; —NR10,
R9 are identical or different optionally branched divalent hydrocarbon radicals,
R10 are identical or different radicals from the group hydrogen or alkyl having 1 to 6 carbon atoms,
R11 are identical or different radicals of the general formula 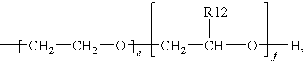
R12 are identical or different alkyl, aryl, or alkaryl radicals having 1 to 30 carbon atoms which optionally comprise ether functions,
e is 0 to 20,
f is 0 to 20,
e+f is >=1,
x is 2 to 18,
a is 2 to 18,
and
A⁻ are identical or different counterions to the positive charges on the quaternary ammonium groups, selected from inorganic or organic anions of the acids HA.

13. The method of claim 8 wherein the formulation is a cosmetic formulation.

14. The method of claim 8 wherein the formulation is a cleaning and care formulation.

15. A cosmetic formulation for protecting animal or human hair against heat damage comprising a polysiloxane containing at least one quaternary ammonium group.

16. The cosmetic formulation of claim 15 wherein the formulation contains the polysiloxane in a concentration of from 0.01 to 20% by mass.

17. The cosmetic formulation of claim 15 wherein the at least one quaternary ammonium group is terminally bonded to the polysiloxane.

18. The cosmetic formulation of claim 15 wherein exactly two quaternary ammonium groups are present and the two quaternary ammonium groups are terminally bonded to the polysiloxane.

19. The cosmetic formulation of claim 15 wherein the polysiloxane is a compound of formula I
[M′Dₙ]₃T Formula I
where
M′=XSiY₂O_{1/2},
D=SiY₂O_{2/2},
T=SiZO_{3/2},
X=identical or different organic radicals which carry quaternary ammonium groups,
Y=identical or different radicals from the group alkyl, aryl, or alkaryl having 1 to 30 carbon atoms,
Z=identical or different radicals from the group alkyl, aryl or alkaryl having 1 to 30 carbon atoms, and
n=2 to 200.

20. The cosmetic formulation of claim 19 wherein X is a radical having the structure —R1-R2, where
R1 are identical or different divalent radicals selected from the group 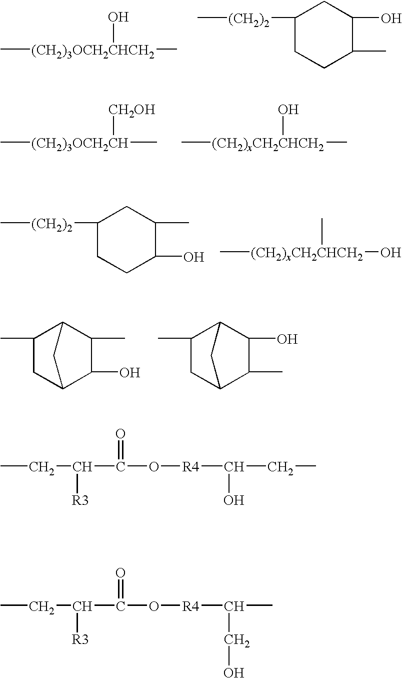
R2 is selected from the group consisting of 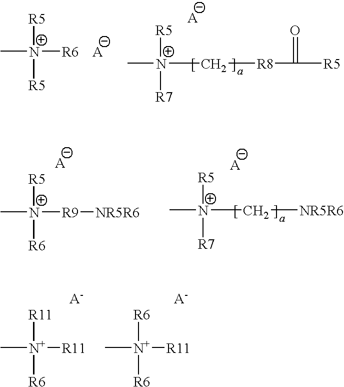
R3 are identical or different radicals from the group hydrogen or alkyl having 1 to 6 carbon atoms,
R4 are identical or different divalent hydrocarbon radicals which optionally comprise ether functions,
R5, R6, R7, in each case independently of one another, are hydrogen or alkyl radicals having 1 to 30 carbon atoms,
R8 are identical or different radicals from the group —O—; —NR10,
R9 are identical or different optionally branched divalent hydrocarbon radicals,
R10 are identical or different radicals from the group hydrogen or alkyl having 1 to 6 carbon atoms,
R11 are identical or different radicals of the general formula 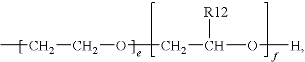
R12 are identical or different alkyl, aryl, or alkaryl radicals having 1 to 30 carbon atoms which optionally comprise ether functions,
e is 0 to 20,
f is 0 to 20,
e+f is >=1,
x is 2 to 18,
a is 2 to 18,
and
A⁻ are identical or different counterions to the positive charges on the quaternary ammonium groups, selected from inorganic or organic anions of the acids HA.
